(19) United States
(12) Patent Application Publication
(10) Pub. No.: US 2012/0252835 A1
(43) Pub. Date: Oct. 4, 2012

(54) STABLE TEMSIROLIMUS COMPOSITION AND PROCESS OF PREPARING SAME

(75) Inventors: Harsh RAJPUT, Ahmedabad (IN); Sandeep MEHTA, Ahmedabad (IN); Pankaj PATEL, Ahmedabad (IN); Bhavesh PATEL, Ahmedabad (IN); Ashish SEHGAL, Ahmedabad (IN); Jayanta Kumar MANDAL, Ahmedabad (IN)

(73) Assignee: ASTRON RESEARCH LIMITED, Ahmedabad (IN)

(21) Appl. No.: 13/431,279

(22) Filed: Mar. 27, 2012

(30) Foreign Application Priority Data
Apr. 1, 2011 (IN) 1124/MUM/2011

(51) Int. Cl. A61K 31/4353; A61P 35/00

(52) U.S. Cl. 514/291

(57) ABSTRACT

The present disclosure describes a stable composition of Temsirolimus for parenteral administration. The composition includes BHA or BHT as anti-oxidants and alcoholic solvent. The pH of the composition is below 5.0. The composition also can include a chelating agent and/or a surfactant. A method of producing the composition is also described.

### FIELD

The present disclosure relates to stable parenteral composition of Temsirolimus and further discloses process for the preparation of the said stable parenteral composition.

### BACKGROUND

Temsirolimus is a hydroxyl ester at the 42^{nd} position of rapamycin. Chemical name of Temsirolimus is (3S,6R,7E,9R,10R,12R,14S,15E,17E,19E,21S,23S,26R,27R,34aS)-9,10,12,13,14,21,22,23,24,25,26,27,32,33,34,34a-Hexa deca hydro-9,27-dihydroxy-3-[(1R)-2-[(1S,3R,4R)-4-hydroxy-3-methoxy cyclohexyl]-1-methyl ethyl]-10,21-dimethoxy-6,8,12,14,20,26-hexa meth 3H-pyrido[2,1-][1,4]oxa azacyclo hentriacontine-1,5,11,28, 29 (4H, 6H, 31H)-pentone 4′-[2,2-bis(hydroxymethyl)propionate]; or Rapamycin, 42-[3-hydroxy-2-(hydroxymethyl)-2-methylpropanoate].

Further, chemical formula of Temsirolimus is C₅₆H₈₇NO₁₆ and has the following chemical structure:

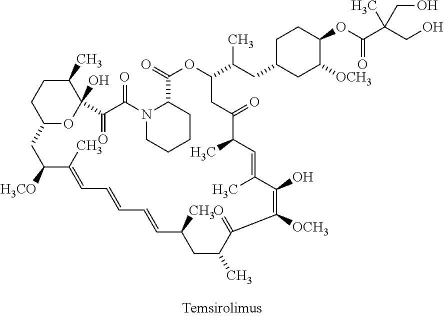

Pharmacologically, Temsirolimus is an anti-neoplastic agent which acts by inhibiting the mammalian target of rapamycin (mTOR), and interferes with the synthesis of proteins that regulate proliferation, growth, and survival of tumor cells.

Temsirolimus has been developed by Wyeth and has been approved in US as TORISEL® for the treatment of advanced renal cell carcinoma. TORISEL® is supplied as a kit consisting of the following:
Product vial (Temsirolimus injection 25 mg/ml) and
Diluent vial.

Apart from Temsirolimus, the Product vial of TORISEL® comprises of d,l-α-tocopherol (0.075% w/v), propylene glycol (50.3% w/v) and dehydrated alcohol (39.5% w/v) as co-solvent, and anhydrous citric acid (0.0025% w/v). Diluent vial comprises of polysorbate 80 (40.0% w/v), polyethylene glycol 400 (42.8% w/v) and dehydrated alcohol (19.9% w/v). Before administration, the Product vial of TORISEL® is diluted with the contents of Diluent vial. This admixture is mixed with 0.9% sodium chloride solution and then administered intravenously to the patient. Experimentally, it is found out that the pH of the Product vial of TORISEL® is in between 6.0 to 7.0, specifically in between 6.0 to 6.5.

Considering the formulation aspect, Temsirolimus is practically insoluble in water but soluble in alcohols which are acceptable for parenteral administration; which include ethanol, propylene glycol, polyethylene glycol and the likes thereof. Temsirolimus has no ionizable functional groups, and its solubility is independent of pH. However, US patent application 2004/0167152 highlights that chemical instability issues persist with these alcoholic solvents. This chemical instability of Temsirolimus is due to oxidative degradation or due to the breaking of the lactone bond, resulting in the formation of the ring opened compounds. Another reason for chemical instability is the precipitation of Temsirolimus upon dilution with an aqueous solvent/diluent.

US2004/0167152 describes that to avert chemical instability of Temsirolimus formulation; anti-oxidants are added optionally in combination with chelating agents. Acceptable anti-oxidants include d,l-α-tocopherol, butylated hydroxyanisole (BHA), butylated hydroxytoluene (BHT), monothioglycerol, ascorbic acid, propyl gallate, and mixtures thereof. Examples of chelating agents included in US2004/0167152 include citric acid, acetic acid, ascorbic acid, ethylene diamine tetra acetic acid (EDTA) or amino acids. According to the US2004/0167152, d,l-α-tocopherol is particularly desirable and used at a concentration of 0.01 to 0.1% w/v with a preferred concentration of 0.075% w/v of the co-solvent concentrate.

US2004/0167152 describes extensively the use of d,l-α-tocopherol as anti-oxidant and citric acid as chelating agent in Temsirolimus composition for parenteral administration. The patent application describes in general that BHA, BHT or their mixtures thereof can be used as anti-oxidants in Temsirolimus composition for parenteral administration.

### SUMMARY

Applicants have found that the use of BHA, BHT or their mixtures thereof as anti-oxidants in Temsirolimus composition does not always give desirable stable compositions. The chemical stability of Temsirolimus composition having BHA and BHT as anti-oxidants is different at different pH ranges. Hence, to obtain a stable Temsirolimus composition for parenteral administration and whose chemical stability can be compared with the commercially available TORISEL®, it is necessary to adjust the pH of the Temsirolimus composition comprising BHA and BHT as anti-oxidants.

This disclosure describes a stable composition of Temsirolimus for parenteral administration, having BHA and BHT as anti-oxidants, wherein the pH of the composition is below 5.0.

This disclosure further describes a method for providing a stable composition of Temsirolimus for parenteral administration, having BHA and BHT as anti-oxidants, wherein the pH of the composition is below 5.0.

This disclosure also describes a chemically stable composition of Temsirolimus whose chemical stability can be compared to commercially available product TORISEL®.

The present disclosure focuses on providing a stable composition of Temsirolimus for parenteral administration, having BHA and BHT as anti-oxidants in the composition; wherein the pH of the composition is below 5.0. The parenteral composition comprises, in addition, co-solvent, solvent and/or chelating agent in the solution. This composition according to the present disclosure can be admixed with infusion solutions, preferably 0.9% sodium chloride injection or 5% dextrose injection prior to parenteral administration.

### DETAILED DESCRIPTION

According to background art, the use of anti-oxidants in the Temsirolimus composition is mandatory to obtain a stable composition; wherein US2004/0167152 describes various anti-oxidants ranging from citric acid, d,l-α-tocopherol, BHA, BHT, monothioglycerol, ascorbic acid, propyl gallate, and mixtures thereof can be used for providing a stable Temsirolimus composition for parenteral administration. The patent application describes in detail about the use of d,l-α-tocopherol as a preferred anti-oxidant for Temsirolimus composition.

Inventors of the present disclosure have studied various anti-oxidants for the preparation of Temsirolimus composition and have found out that use of anti-oxidants is not the only criteria in obtaining stable compositions of Temsirolimus. Experimentally, it was found that pH of the Product vial of marketed product TORISEL® is in between 6.0 to 7.0; particularly in between 6.0 to 6.5. Inventors of the present invention have also found experimentally that use of monothioglycerol as an anti-oxidant in the Temsirolimus composition does not provide a stable composition at any pH. Specifically, when BHA, BHT or mixtures thereof are used as anti-oxidants in Temsirolimus composition, it is found that stable composition is only obtained when the pH of the composition is below 5.0; preferably the pH of the composition should be in a range between 3.5 to 5.0.

The term “stable composition” generally relates to the maintained effectiveness of the active ingredient in the composition. According to the present disclosure, a stabilized composition shows physical and chemical stability when exposed to stability conditions of 2-8° C. or 25° C. for at least 3 months.

Physical stability, according to the present disclosure, is characterized as existence of the said composition as a clear colorless solution. Chemical stability, according to the present disclosure, is characterized as 1) assay percentage of Temsirolimus in the said composition, wherein the assay of Temsirolimus measured by HPLC does not vary by more than 10% of the initial assay percentage; and 2) amount of Total Impurities (excluding Impurity C) is not more than 3%.

According to the present disclosure, an assay of Temsirolimus is performed by HPLC method which is well known in to a person skilled in the art.

According to the present disclosure, concentration of BHA is in the range of 0.0002 to 0.0005% and BHT is in the range of 0.0009 to 0.002% of the composition. When used in combination, preferable concentration of BHA is 0.0003% and BHT is 0.001% of the composition of Temsirolimus.

The composition of Temsirolimus comprises of, apart from Temsirolimus and BHA and BHT as anti-oxidants, a co-solvent system comprising alcoholic solvent, a diluent system comprising of a diluent solvent. The composition of Temsirolimus may further contain chelating agent and/or surfactant.

According to the present disclosure, alcoholic solvent may contain one or more alcohols which can be used as pharmaceutically acceptable solvents comprising of ethanol, polyethylene glycol, propylene glycol or the likes thereof.

According to the present disclosure, diluent solvent may include but not limiting to water, ethanol, polyethylene glycols, propylene glycols and the likes thereof.

According to one of the embodiments, the concentration of Temsirolimus in the composition of Temsirolimus can be in the range of 0.05 mg/ml to 50 mg/ml, preferably 25 mg/ml.

In preferred embodiments according to the present disclosure, chelating agent is further added in the composition of Temsirolimus. Examples of chelating agent used may include citric acid, ascorbic acid, acetic acid, EDTA or the likes thereof in the range of 0.001 to 0.01% w/v of the composition.

In certain embodiments, the surfactant may comprise of conventionally used parenterally acceptable surfactants. Conventionally used surfactants include but are not limited to polysorbates, bile acids (optionally used with lecithins), ethoxylated vegetable oils, and the likes thereof.

In another embodiment, the composition of Temsirolimus is prepared by admixture of the contents of product vial comprising Temsirolimus, BHA, BHT, co-solvent system and chelating agents with contents of diluent vial comprising diluent solvent and optional surfactant.

In one of the embodiments, the composition of Temsirolimus as disclosed in the present disclosure can also be provided as a one vial composition comprising Temsirolimus, BHA, BHT, co-solvent system, diluent system, chelating agent and optionally surfactant.

According to the present disclosure, composition of Temsirolimus can be admixed with infusion solutions, preferably 0.9% sodium chloride injection or 5% dextrose injection prior to parenteral administration.

During the process for the preparation of composition of Temsirolimus, inert air sparging is continued during any of the desired stages of the process to remove dissolved oxygen from the composition. Inert gases include nitrogen, argon and related gases.

### EXAMPLE

The present disclosure has been described by way of example only. It is to be recognized that modifications falling within the scope and spirit of the claims, which would be obvious to a person skilled in the art based upon the disclosure herein, are also considered to be included within the scope of this disclosure.

Generalized composition and procedure:

Temsirolimus Injection 25 mg/mL Kit

| | | |
|---|---|---|
| Sr. no. | Ingredient | Amount in range |

PRODUCT VIAL

| | | |
|---|---|---|
| 1. | Temsirolimus | 0.05-50 mg/ml |
| 2. | Co-solvent system | q.s. |
| 3. | Chelating agent | 0.001%-0.01% |
| 4. | Anti-oxidants (BHA) | 0.0002%-0.0005% |
| 5. | Anti-oxidants (BHT) | 0.0009%-0.002% |
| 6. | Nitrogen | q.s. to sparge |

DILUENT VIAL

| | | |
|---|---|---|
| 1. | Diluent solvent | q.s. |
| 2. | Surfactant | q.s. |
| 3. | Nitrogen | q.s. to sparge |

Manufacturing procedure:

Product VIAL
a) Take batch quantity of alcoholic solvent or mixture of alcoholic solvents into a vessel.
b) Dissolve chelating agent into alcoholic solvent and add the same into the solution of step a).
c) Dissolve BHA into alcoholic solvent and add the same to solution of step b).
d) Dissolve BHT into alcoholic solvent and add the same to solution of step c).
e) Add batch quantity of Temsirolimus into solution of step d) and stir for 15-30 minutes to dissolve the same completely.
f) Compensate the alcohol solvent and stir.
g) Filter the bulk solution through 0.22 micron sterile sterilizing grade filter and collect the solution into sterile collection vessel.
h) Fill the solution into sterile suitable containers.

Diluent vial:
a) Take batch quantity of surfactant and dissolve in diluent solvent
b) Optionally sparge the solution obtained in step a) during the process.
c) Filter the bulk solution through 0.22 micron sterile sterilizing grade filter and collect the solution into sterile collection vessel.
d) Fill the solution into sterile suitable containers.

### Example 1

Temsirolimus Injection 25 mg/mL Kit

| | | |
|---|---|---|
| Sr. no. | Ingredient | Amount in mg/ml |

PRODUCT VIAL

| | | |
|---|---|---|
| 1. | Temsirolimus | 25.0 |
| 2. | Propylene Glycol | 503.0 |
| 3. | Dehydrated Alcohol | 395.0 |
| 4. | Citric acid anhydrous | 0.025 |
| 5. | BHA | 0.003 |
| 6. | BHT | 0.01 |
| 7. | Nitrogen | q.s. to sparge |

DILUENT VIAL

| | | |
|---|---|---|
| 1. | Polyethylene Glycol 400 | 428.0 |
| 2. | Polysorbate 80 | 400.0 |
| 3. | Dehydrated Alcohol | 199.0 |
| 4. | Nitrogen | q.s. to sparge |

Manufacturing procedure:

Product vial
a) Take batch quantity of Propylene Glycol into suitable vessel.
b) Add batch qty of dehydrated alcohol into Step a).
c) Dissolve citric acid into dehydrated alcohol and add the same into the solution of step b).
d) Dissolve BHA into dehydrated alcohol and add the same to solution of step c).
e) Dissolve BHT into dehydrated alcohol and add the same to solution of step d).
f) Add batch qty of Temsirolimus API into solution of step e) and stir to dissolve the same completely.
g) Compensate the evaporated dehydrated alcohol.
h) Filter the bulk solution through 0.22 micron sterile sterilizing grade filter and collect the solution into sterile collection vessel.
i) Fill the solution into sterile suitable container.

Diluent vial:
a) Take batch quantity of polysorbate 80 and dissolve in diluent solvent
b) Optionally sparge the solution obtained in step a) during the process.
c) Filter the bulk solution through 0.22 micron sterile sterilizing grade filter and collect the solution into sterile collection vessel.
d) Fill the solution into sterile suitable containers.

As per the requirement, the manufacturing process can be modified to include sparging with inert gas at any stage of the process.

The pH of the product vial is 4.6.

### Stability Study for Example 1:

| | | | | | |
|---|---|---|---|---|---|
| | 40° C. | 25° C. | 25° C. | 2-8° C. | 2-8° C. |
| | 21 | 21 | 3 | 21 | 3 |
| Initial | Days | Days | Months | Days | Months |

Test parameters

| | | | | | | |
|---|---|---|---|---|---|---|
| Description | CCS | CCS | CCS | CCS | CCS | CCS |
| Assay (HPLC) | 102.6 | 100.8 | 103.2 | 94.7 | 102.3 | 101.8 |
| Ph | 4.6 | 4.3 | 4.3 | 4.3 | 4.3 | 4.4 |

Related substances

| | | | | | | |
|---|---|---|---|---|---|---|
| Sirolimus | 0.549 | 0.433 | 0.522 | 0.263 | 0.632 | 0.361 |
| Isomer-C | 3.425 | 7.358 | 5.584 | 6.781 | 3.167 | 4.049 |
| Imp at RRT | 0.137 | 0.413 | 0.264 | 0.396 | 0.209 | 0.391 |
| 0.85 | | | | | | |
| Imp at RRT | 0.033 | 0.069 | 0.049 | ND | 0.04 | ND |
| 0.87 | | | | | | |
| Single max. | 0.079 | 0.104 | 0.105 | 0.074 | 0.100 | 0.085 |
| unknown | | | | | | |
| impurity | | | | | | |
| Total Imp. | 1.075 | 1.348 | 1.119 | 1.005 | 1.153 | 1.006 |
| (excluding | | | | | | |
| Isomer C) | | | | | | |

CCS: Clear colorless solution

### Example 2

Temsirolimus Injection 25 mg/mL Kit

| | | |
|---|---|---|
| Sr. no. | Ingredient | Amount in mg/ml |

PRODUCT VIAL

| | | |
|---|---|---|
| 1. | Temsirolimus | 25.0 |
| 2. | Propylene Glycol | 503.0 |
| 3. | Dehydrated Alcohol | 395.0 |
| 4. | Citric acid anhydrous | 0.025 |
| 5. | BHA | 0.003 |
| 6. | BHT | 0.01 |
| 7. | NaOH | q.s. to adjust pH till 6.5 |
| 8. | Nitrogen | q.s. to sparge |

DILUENT VIAL

| | | |
|---|---|---|
| 1. | Polyethylene Glycol 400 | 428.0 |
| 2. | Polysorbate 80 | 400.0 |
| 3. | Dehydrated Alcohol | 199.0 |
| 4. | Nitrogen | q.s. to sparge |

Manufacturing procedure:

Product vial
a) Take batch quantity of Propylene Glycol into suitable vessel.
b) Add batch qty of dehydrated alcohol into Step a).
c) Dissolve citric acid into dehydrated alcohol and add the same into the solution of step b).
d) Dissolve BHA into dehydrated alcohol and add the same to solution of step c).
e) Dissolve BHT into dehydrated alcohol and add the same to solution of step d).
f) Add batch qty of Temsirolimus API into solution of step e) and stir to dissolve the same completely.
g) Adjust the pH of the composition to 6.5 using NaOH.
h) Compensate the evaporated dehydrated alcohol.
i) Filter the bulk solution through 0.22 micron sterile sterilizing grade filter and collect the solution into sterile collection vessel.
j) Fill the solution into sterile suitable container.

Diluent vial:
a) Take batch quantity of polysorbate 80 and dissolve in diluent solvent
b) Optionally sparge the solution obtained in step a) during the process.
c) Filter the bulk solution through 0.22 micron sterile sterilizing grade filter and collect the solution into sterile collection vessel.
d) Fill the solution into sterile suitable containers.

As per the requirement, the manufacturing process can be modified to include sparging with inert gas at any stage of the process.

The pH of the product vial is 6.5.

### Stability Study for Example 2:

| | | |
|---|---|---|
| | 40° C., | 25° C. |
| Initial | 7 Days | 7 Days |

Test parameters

| | | | |
|---|---|---|---|
| Description | CCS | CCS | CCS |
| Assay (HPLC) | 101.4% | 85.5% | 88.5% |
| pH | | | |

Related substances

| | | | |
|---|---|---|---|
| Sirolimus | 0.459% | 0.160% | 0.228% |
| Isomer-C | 4.639% | 5.312% | 5.881% |
| Imp at RRT 0.85 | 0.336% | 0.308% | 0.297% |
| Imp at RRT 0.87 | 0.183% | ND | 0.021% |
| Single max. unknown | 0.083% | 2.390% | 1.232% |
| impurity | | | |
| Total Imp. | 1.307% | 6.094% | 4.119% |
| (Excluding Isomer C) | | | |

CCS: Clear Colourless solution

As the level of impurities generated with decomposition of Temsirolimus at seven days at 25° C. are very significant as compared to the level observed in product as in example 1, further testing of product as in example 2 were discontinued. Further time-points/stability conditions kept on stability were at 2-8° C., 25° C. and 40° C., were also discontinued after seven days as the results were very discouraging.

On comparing the results obtained in example 1 and 2, it is clear that the assay percentage of Temsirolimus has reduced in the composition. Further, there is a drastic increase of total impurity levels in the composition. The results of example 1 and 2 indicate that when BHA/BHT or combinations thereof are used in the Temsirolimus composition as anti-oxidants, a stable composition can be obtained when the pH of the composition is below 5.0, preferably in between 3.5-5.0.

Further examples 3 and 4 provides the composition of Temsirolimus having monothioglycerol as anti-oxidant in the composition; wherein example 3 discloses a composition having pH less than 5.0 and example 4 discloses a composition having pH more than 5.0.

### Example 3

Temsirolimus Injection 25 mg/mL Kit

| | | |
|---|---|---|
| Sr. no. | Ingredient | Amount in mg/ml |

PRODUCT VIAL

| | | |
|---|---|---|
| 1. | Temsirolimus | 25.0 |
| 2. | Propylene Glycol | 503.0 |
| 3. | Dehydrated Alcohol | 395.0 |
| 4. | Citric acid anhydrous | 0.025 |
| 5. | Monothioglycerol | 10 |
| 6. | Nitrogen | q.s. to sparge |

DILUENT VIAL

| | | |
|---|---|---|
| 1. | Polyethylene Glycol 400 | 428.0 |
| 2. | Polysorbate 80 | 400.0 |
| 3. | Dehydrated Alcohol | 199.0 |
| 4. | Nitrogen | q.s. to sparge |

Manufacturing procedure:

Product vial
a) Take batch quantity of Propylene Glycol into suitable vessel.
b) Add batch qty of dehydrated alcohol into Step a).
c) Dissolve citric acid into dehydrated alcohol and add the same into the solution of step b).
d) Dissolve monothioglycerol into dehydrated alcohol and add the same to solution of step c).
e) Add batch quantity of Temsirolimus API into solution of step d) and stir to dissolve the same completely.
f) Compensate the evaporated dehydrated alcohol.
g) Filter the bulk solution through 0.22 micron sterile sterilizing grade filter and collect the solution into sterile collection vessel.
h) Fill the solution into sterile suitable container.

Diluent vial:
a) Take batch quantity of polysorbate 80 and dissolve in diluent solvent
b) Optionally sparge the solution obtained in step a) during the process.
c) Filter the bulk solution through 0.22 micron sterile sterilizing grade filter and collect the solution into sterile collection vessel.
d) Fill the solution into sterile suitable containers.

As per the requirement, the manufacturing process can be modified to include sparging with inert gas at any stage of the process.

The pH of the product vial is below 5.0, specifically 4.4.

### Stability Study of Example 3:

| | | |
|---|---|---|
| | 40° C., | 25° C. |
| Initial | 7 Days | 7 Days |

Test parameters

| | | | |
|---|---|---|---|
| Description | CCS | CCS | CCS |
| Assay (HPLC) | 99.0 | 85.6 | 88.3 |
| pH | 4.4 | 4.4 | 4.7 |

Related substances

| | | | |
|---|---|---|---|
| Sirolimus | 0.508 | 0.279 | 0.394 |
| Isomer-C | 3.332 | 5.049 | 3.247 |
| Imp at RRT1.36 | ND | ND | ND |
| Imp at RRT 0.85 | 0.133 | 1.011 | 0.679 |
| Imp at RRT 0.87 | 0.040 | 1.089 | 0.797 |
| Single max. unknown | 0.082 | 0.363 | 0.459 |
| impurity | | | |
| Total Imp. | 1.298 | 3.057 | 2.612 |
| (Excluding Isomer C) | | | |

CCS: Clear colorless solution

Stability studies for example 3, wherein monothioglycerol is used as an anti-oxidant in the product vial of Temsirolimus composition shows that the assay percentage of Temsirolimus has reduced to unacceptable amounts. Hence these results shows that Temsirolimus composition have monothioglycerol as an anti-oxidant and having a pH of less than 5.0 does not provide a stable composition of Temsirolimus.

As the level of impurities generated with decomposition of Temsirolimus even at seven days is very significant as compared to the levels observed in example 1, the further testing was discontinued. Further time-points/stability conditions kept on stability were at 2-8° C., 25° C. and 40° C., were discontinued after seven days as the results were very discouraging.

### Example 4

Temsirolimus Injection 25 mg/mL Kit

| | | |
|---|---|---|
| Sr. no. | Ingredient | Amount in mg/ml |

PRODUCT VIAL

| | | |
|---|---|---|
| 1. | Temsirolimus | 25.0 |
| 2. | Propylene Glycol | 503.0 |
| 3. | Dehydrated Alcohol | 395.0 |
| 4. | Citric acid anhydrous | 0.025 |
| 5. | Monothioglycerol | 10 |
| 6. | Nitrogen | q.s. to sparge |

DILUENT VIAL

| | | |
|---|---|---|
| 1. | Polyethylene Glycol 400 | 428.0 |
| 2. | Polysorbate 80 | 400.0 |
| 3. | Dehydrated Alcohol | 199.0 |
| 4. | Nitrogen | q.s. to sparge |

Manufacturing procedure:

Product vial
a) Take batch quantity of Propylene Glycol into suitable vessel.
b) Add batch qty of dehydrated alcohol into Step a).
c) Dissolve citric acid into dehydrated alcohol and add the same into the solution of step b).
d) Dissolve monothioglycerol into dehydrated alcohol and add the same to solution of step c).
e) Add batch quantity of Temsirolimus API into solution of step d) and stir to dissolve the same completely.
f) Adjust the pH to 6.5 using NaOH.
g) Compensate the evaporated dehydrated alcohol.
h) Filter the bulk solution through 0.22 micron sterile sterilizing grade filter and collect the solution into sterile collection vessel.
i) Fill the solution into sterile suitable container.

Diluent vial:
a) Take batch quantity of polysorbate 80 and dissolve in diluent solvent
b) Optionally sparge the solution obtained in step a) during the process.
c) Filter the bulk solution through 0.22 micron sterile sterilizing grade filter and collect the solution into sterile collection vessel.
d) Fill the solution into sterile suitable containers.

As per the requirement, the manufacturing process can be modified to include sparging with inert gas at any stage of the process.

The pH of the product vial is above 5.0, specifically 6.2.

### Stability Study of Example 4:

| | | |
|---|---|---|
| | 40° C. | 25° C. |
| Initial | 7 Days | 7 Days |

Test parameters

| | | | |
|---|---|---|---|
| Description | CCS | CCS | CCS |
| Assay (HPLC) | 98.8 | 89.4 | 95.6 |
| pH | 6.2 | 5.4 | 5.6 |

Related substances

| | | | |
|---|---|---|---|
| Sirolimus | 0.559 | 0.402 | 0.340 |
| Isomer-C | 5.742 | 7.353 | 7.057 |
| Imp at RRT1.36 | ND | ND | ND |
| Imp at RRT 0.85 | 0.117 | 0.590 | 0.608 |
| Imp at RRT 0.87 | 0.234 | 0.352 | 0.440 |
| Single max. unknown | 0.146 | 4.746 | 1.407 |
| impurity | | | |
| Total Imp. | 1.423 | 11.520 | 5.187 |
| (Excluding Isomer C) | | | |

CCS: Clear colorless solution

Stability studies for example 4, wherein monothioglycerol is used as an anti-oxidant in the product vial of Temsirolimus composition shows that the assay percentage of Temsirolimus has comparatively reduced. Further there is a drastic increase in the total impurity levels in the product vial. Hence these results shows that Temsirolimus composition have monothioglycerol as an anti-oxidant and having a pH of more than 5.0 does not provide a stable composition of Temsirolimus.

As the levels of impurities generated with decomposition of Temsirolimus even at seven days are very significant as compared to the level observed in example 1, further testing was discontinued. Further time-points/stability conditions kept on stability were at 2-8° C., 25° C. and 40° C., were discontinued after seven days as the results were very discouraging.

### Example 5

Temsirolimus Injection 10 mg/mL single vial

| | | |
|---|---|---|
| Sr. no. | Ingredient | Amount in mg/ml |

SINGLE VIAL

| | | |
|---|---|---|
| 1. | Temsirolimus | 10.0 |
| 2. | Dehydrated Alcohol | 277.4 |
| 3. | Propylene glycol | 201.2 |
| 4. | Polyethylene glycol 400 | 256.8 |
| 5. | Polysorbate 80 | 240.0 |
| 6. | Anhydrous citric acid | 0.01 |
| 7. | Butylated hydroxy anisole | 0.0012 |
| 8. | Butylated hydroxy toluene | 0.004 |
| 9. | Nitrogen | q.s. to sparge |

Manufacturing procedure:
a) Take propylene glycol into suitable vessel.
b) Add dehydrated alcohol into propylene glycol and mix well.
c) Add polyethylene glycol 400 into the solution obtained in step b with continuous stirring.
d) Add Polysorbate 80 into the solution obtained in step c with continuous stirring.
e) Add anhydrous citric acid into the solution obtained in step d with continuous stirring.
f) Add Butylated hydroxy anisole and Butylted hydroxy toluene to the solution obtained in step e.
g) Add Temsirolimus into the solution obtained in step f and mix well.

As per the requirement, the manufacturing process can be modified to include sparging with inert gas at any stage of the process.

The pH of the single vial product is 4.1.

### Stability Study for Example 5:

| | | | | |
|---|---|---|---|---|
| | 25° C. | 25° C. | 2-8° C. | 2-8° C. |
| Initial | 2 M | 3 M | 1 M | 3 M |

Test parameters

| | | | | | |
|---|---|---|---|---|---|
| Description | CCS | CCS | CCS | CCS | CCS |
| Assay (HPLC) | 93.0 | 92.3 | 90.7 | 90.1 | 91.2 |
| pH | 4.1 | 3.73 | 3.8 | NA | 3.8 |

Related substances

| | | | | | |
|---|---|---|---|---|---|
| Sirolimus | 0.268 | 0.046 | 0.519 | 0.206 | 0.132 |
| Isomer-C | 5.067 | 6.291 | 5.851 | 6.064 | 6.07 |
| Imp at RRT 0.85 | 0.693 | 0.691 | 0.734 | 0.753 | 0.776 |
| Imp at RRT 0.87 | ND | ND | ND | ND | ND |
| Single max. unknown | 0.172 | 0.342 | 0.215 | 0.172 | 0.182 |
| impurity | | | | | |
| Total Imp. | 1.322 | 1.612 | 2.084 | 1.401 | 1.434 |
| (excluding Isomer C) | | | | | |

CCS: Clear colorless solution
NA—Not analyzed

Stability studies for example 5 (Temsirolimus injection single vial) describes that the single vial composition comprising BHA and BHT as anti-oxidants and having pH less than 5 is stable according to the present invention.

While the disclosed composition and method have been described in conjugation with a preferred embodiment, it will be apparent to one skilled in the art that other objects and refinements of the disclosed compositions and method may be made within the purview and scope of the disclosure.

The disclosure, in its various aspects and disclosed forms, is well adapted to the attainment of the stated objects and advantages of others. The disclosed details are not to be taken as limitations on the claims.

What is claimed is:

1. A stable pharmaceutical composition of Temsirolimus for parenteral administration, comprising:
Temsirolimus as an active pharmaceutical ingredient (API);
at least one anti-oxidant selected from the group consisting of butylatedhydroxyanisole (BHA) and butylatedhydroxytoluene (BHT); and
an alcoholic solvent,
wherein a pH of the composition is below about 5.0.

2. The composition according to claim 1, wherein the pH of said composition is more than 3.5.

3. The composition according to claim 1, wherein the API is present in the composition in an amount of about 0.05 mg/ml to about 50 mg/ml.

4. The composition according to claim 1, wherein the at least one anti-oxidant is present in an amount of about 0.0002 to about 0.002% of the composition.

5. The composition according to claim 1, wherein the alcoholic solvent is at least one selected from the group comprising ethanol, polyethylene glycol and propylene glycol.

6. The composition according to claim 1, further comprising a diluent solvent.

7. The composition according to claim 6, wherein the diluent solvent is at least one selected from the group comprising water, ethanol, polyethylene glycol, propylene glycol and polysorbates.

8. The composition according to claim 1, further comprising a chelating agent.

9. The composition according to claim 8, wherein the chelating agent is at least one selected from the group comprising citric acid, ascorbic acid, acetic acid and ethylene diaminetetraacetic acid (EDTA).

10. The composition according to claim 1, further comprising a surfactant.

11. The composition according to claim 10, wherein the surfactant is at least one selected from the group comprising a bile acid, ethoxylated vegetable oil, and polysorbates.

12. The composition according to claim 1, wherein the composition is stable such that an assay percentage of Temsirolimus after storage of the composition does not vary by more than 10% of an initial assay percentage, and an amount of total impurities excluding Isomer C in the composition is not more than 3% after storage of the composition.

13. The composition according to claim 12, wherein the storage conditions are 2-8° C. or 25° C. for a time period of at least three months.

14. The composition according claim 1, wherein the composition is stable such that the composition is a clear and colorless solution after storage of the composition.

15. The composition according to claim 14, wherein the storage conditions are 2-8° C. or 25° C. for a time period of at least three months.

16. The composition according to claim 1, further comprising an infusion solution.

17. The composition according to claim 16, wherein the infusion solution is at least one selected from the group consisting of sodium chloride and dextrose.

18. A process for preparing a stable pharmaceutical composition of Temsirolimus for parenteral administration, comprising:
a) preparing a solution of an anti-oxidant in a co-solvent system comprising an alcoholic solvent, the anti-oxidant being at least one selected from the group consisting of butylatedhydroxyanisole (BHA) and butylatedhydroxytoluene (BHT) and the alcoholic solvent being at least one selected from the group comprising ethanol, polyethylene glycol and propylene glycol; and
b) adding Temsirolimus and optionally a chelating agent to the solution prepared in (a), the chelating agent being at least one selected from the group comprising citric acid, ascorbic acid, acetic acid and ethylenediaminetetraacetic acid (EDTA),
wherein a pH of the solution obtained in (b) is below about 5.0.

19. The process according to claim 18, comprising mixing a diluent solvent that is at least one selected from the group comprising water, ethanol, polyethylene glycol, propylene glycol and polysorbates.
